(19) United States
(12) Patent Application Publication
(10) Pub. No.: US 2005/0221501 A1
(43) Pub. Date: Oct. 6, 2005

(54) Dissolution method

(76) Inventors: Kate Isabel Arnot, Cheshire (GB); Donal Joseph Murphy, Cheshire (GB)

Correspondence Address: FISH & NEAVE IP GROUP;ROPES & GRAY LLP, ONE INTERNATIONAL PLACE, BOSTON, MA, 02110-2624, US

(21) Appl. No.: 10/489,213

(22) PCT Filed: Dec. 24, 2003
(86) PCT No.: PCT/GB03/05667

§ 371 Date: Apr. 15, 2005

(51) Int. Cl. G01N 33/00

(52) U.S. Cl. 436/98

(57) ABSTRACT

A method for measuring the release of 4-(3′-chloro-4′-fluoroanilino)-7-methoxy-6-(3-morpholinopropoxy)quinazoline or a pharmaceutically-acceptable salt thereof (the Agent) from a pharmaceutical composition, which method comprises: (i) immersing a pharmaceutical composition containing the Agent in a dissolution medium, wherein the dissolution medium comprises water and a non-ionic surfactant; and (ii) determining the concentration of the Agent in the dissolution medium at one or more time points following immersion of the pharmaceutical composition. The method is suitable for monitoring batch-to-batch variability as part of a quality control procedure; or as a means for determining the bioequivalence of different formulations containing the Agent.

This application is a national stage filing under 35 U.S.C. 371 of International Application No. PCT/GB03/005667, filed Dec. 24, 2003, the specification of which is incorporated by reference herein. International Application No. PCT/GB02/04047 was published under PCT Article 21(2) in English.

The present invention relates to a method for measuring the release of a pharmacologically active agent from a pharmaceutical composition, more particularly to a method for measuring the release of 4-(3′-chloro-4′-fluoroanilino)-7-methoxy-6-(3-morpholinopropoxy)quinazoline or a pharmaceutically-acceptable salt thereof (hereinafter referred to as the “Agent”) from a pharmaceutical composition such as a solid instant release composition such as a tablet or capsule.

The Agent is disclosed in International Patent Application WO 96/33980 (Example 1) and is a potent inhibitor of the epidermal growth factor receptor (EGFR) family of tyrosine kinase enzymes such as erbB1. The Agent has the structure of the Formula I 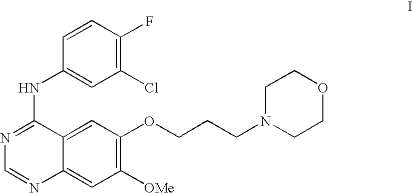
and is now known as Iressa (registered trade mark), gefitinib (Unites States Adopted Name), by way of the code number ZD1839 and Chemical Abstracts Registry Number 184475-35-2.

The Agent possesses anti-proliferative activity such as anti-cancer activity and, accordingly, is useful in methods of treatment of proliferative disease such as cancer in the human or animal body. The Agent is expected to be useful in the treatment of diseases or medical conditions mediated alone or in part by EGFR receptor tyrosine kinases, particularly cancers such as breast, lung, colon, rectum, stomach, prostate, bladder, pancreas and ovary and head and neck cancers.

The Agent is a weakly basic compound and has two basic groups with pKₐ's of 5.3 and 7.2. Consequently, the solubility of the Agent is highly dependent upon pH. The free-base form of the Agent is soluble at pH 1 (10 to 30 ml of aqueous solvent required to dissolve 1 g of Agent) but has very low solubility above pH 7, with the solubility dropping sharply between pH 4 and pH 6 (≧10000 ml of aqueous solvent required to dissolve 1 g of Agent at pH 6).

The pH values between which the agent shows the greatest change in solubility (pH 5 to 6) corresponds approximately to the pH range in the regions of the GI tract from which the Agent is thought to be absorbed. The Agent must be in solution in the GI tract in order to be absorbed, therefore even small variations in the concentration of the Agent at the site of absorption may have a marked effect upon the pharmacokinetic properties of the Agent, such as rate and extent of absorption and bioavailability as a result of the pH sensitive solubility profile of the Agent.

There is therefore a need to ensure that pharmaceutical formulations containing the Agent are carefully manufactured to ensure that the Agent is delivered in a consistent manner to minimise any variability in the pharmacokinetic properties of the Agent such as the Cₘₐₓ, AUC or bioavailability of the Agent. Whilst in-vivo testing is essential to evaluate the pharmacokinetic properties of a pharmaceutical formulation such in-vivo tests are not practical for routinely assessing, for example batch-to-batch variability for quality control purposes, the storage stability of formulations or the effect of formulation modifications on bioequivalence between different formulations containing the Agent.

Numerous in-vitro dissolution tests have been developed in an attempt to correlate in-vitro dissolution of a drug with its in-vivo behaviour. United Sates Pharmacopoeia 24 (USP 24), section <711> describes suitable in-vitro tests and apparatus for measuring in-vitro dissolution profiles from pharmaceutical formulations. Typically the apparatus described in USP 24 comprise an agitated dissolution vessel containing a suitable dissolution medium into which a pharmaceutical composition is placed. The dissolution medium is then periodically sampled to determine the quantity of drug in solution. USP 24, section <1088> describes suitable dissolution media for assessing immediate release dosage forms in dissolution apparatus. A preferred USP dissolution medium for basic drugs such as the Agent is 0.1N buffered hydrochloric acid. The USP indicates that other media may be used if substantiated by the solubility characteristics of the drug such as a buffered aqueous solution (typically pH 4 to 8).

The general guidance document issued by the U.S. Department of Health and Human Services, Food and Drug Administration, Center for Drug Evaluation and Research (CDER), August 1997, entitled “Guidance for Industry: Dissolution Testing of Immediate Release Solid Oral Dosage Forms” indicates that the mean (T50%) gastric residence (emptying) time is 15-20 minutes under fasting conditions. Based on this information, a conservative conclusion is that a drug product which undergoes 85% dissolution in 15 minutes under mild dissolution test conditions in a 0.1N HCl dissolution medium will behave like a solution and generally should not have any bioavailability problems.

Noory et al. (Dissolution Technologies February 2000, pp 16-18) describe a step-wise procedure for developing a suitable dissolution test for sparingly water-soluble drugs. In step 1 of the procedure described Noory states that the effect of pH of the dissolution medium should be evaluated, by performing a dissolution test in one of the standard dissolution media described in the USP, particularly 0.1N HCl, pH 4.5 sodium acetate buffered medium and pH 6.8 phosphate buffered medium. Noory states that the preferred dissolution medium should be selected on the basis of this initial screening test. Noory then suggests using a surfactant in those cases where the drug exhibits poor solubility in all of these standard media.

The Agent exhibits a high solubility at low pH and is highly soluble at pH 1. Accordingly when immediate release formulations containing the Agent were tested using dissolution media with pH's of 1.2 and 3.0 complete dissolution was observed within 15 minutes. In view of the high solubility in these media it was expected, based upon Noory and the FDA guidance document discussed above, that a USP standard acidic dissolution medium (such as 0.1N HCl) would provide a suitable dissolution medium for testing formulations containing the Agent. However, these conventional acidic dissolution media failed to discriminate between formulations containing the Agent which were known to exhibit different pharmacokinetic properties when administered in-vivo.

Noory et al suggest that in those cases where a drug will not dissolve in one of the standard USP dissolution media that a small amount of surfactant may be used to aid dissolution. Noory indicates that the lowest amount of surfactant possible to solubilise the drug to give 85% dissolution in 120 minutes is used. Typically this was a surfactant concentration of 2% or less. Generally, the surfactant used in Noory was the anionic surfactant sodium lauryl sulfate.

Amidon et al. (Pharmaceutical Research Vol. 12, 3, 1995, 413-420) suggest the use of surfactants such as sodium lauryl sulfate for water insoluble drugs. Amidon indicates that it is important that the in-vitro dissolution medium should represent as closely as possible the in-vivo dissolution medium.

Shah et. al. (Pharmaceutical Research Vol 6, No. 7, 1989 612-618) describes an in-vitro dissolution test for water-insoluble drugs using a dissolution medium containing a surfactant. Sodium lauryl sulfate was the preferred surfactant as this was found to provide the best solubilisation properties. Increasing surfactant concentration resulted in an increased dissolution of the drug and, in the case of the drug Griseofluvin, an aqueous dissolution medium containing 4% sodium lauryl sulfate gave at least 75% dissolution in 60 minutes.

We have found that in immediate release pharmaceutical formulations containing the Agent 100% of the Agent was dissolved in less than 60 minutes using a dissolution medium containing 0.5% v/v or more sodium lauryl sulfate. However, despite the high solubility in this dissolution medium, the test was unable to discriminate between different formulations containing the Agent which were known to exhibit different in-vivo pharmacokinetic profiles.

WO 98/20340 describes a dissolution test for measuring the release of a steroid from a solid pharmaceutical composition. The method uses a an aqueous dissolution medium containing from 0.0025 to 0.15% w/v of Polysorbate 20.
Abrahamsson et al. (Pharmaceutical Research, 1994, 11, 1093-1097) discusses the effect of surfactants upon the release of felodipine from an extended release formulation in a number of different dissolution media. Abrahamsson found that the release of felodipine was almost the same in 0.1N HCl as in phosphate buffer at pH6.5. Abrabamsson also suggests for the particular felodipine extended release formulation tested, sodium lauryl sulfate was a less suitable surfactant, probably as a result of an interaction between this surfactant and the HPMC extended release matrix.
Nagata et al. (Yakugaku Zasshi (1979), 99(10), 965-70) review the properties of commercial phytonadion tablets and the effect of Polysorbate 80 on the dissolution behaviour of the tablets.

Dressman (Drugs in Pharmaceutical Sciences (2000), 106 (oral absorption, 151-181, ISSN:0360-2583) reviews suitable dissolution tests for a range of drugs. In relation to the use of synthetic surfactants such as Tween™ or sodium lauryl sulfate, Dressman indicates that the dissolution medium should be selected so as to replicate as closely as possible the in-vivo environment and suggests that bile salts and other bile components may be used in a dissolution medium. Dressman also mentions the possibility of using synthetic surfactants instead of bile components, however, Dressman indicates that the such surfactants may not accurately replicate the in-vivo environment and indicates that much research is still required to identify a synthetic surfactant that could be used as general substitute for natural surfactants found in bile.

Chen et al. (Pharmaceutical Research (2003), 20(5), 797-801) review the dissolution behaviour of a 2-(3H)-benzoxazolone compound in the presence of Tween 80.

There remains a need for a discriminatory in-vitro test that is sufficiently sensitive to be able to demonstrate bioequivalence between different formulations containing the Agent or between different batches of the same formulation.

We have surprisingly found that certain dissolution media containing non-ionic surfactant provide the means for a highly discriminatory dissolution test that is able to detect, in-vitro, differences between formulations containing the Agent that are predictive of in-vivo bioequivalence or bio-inequivalence.

According to a first aspect of the present invention there is provided a method for measuring the release of 4-(3′-chloro-4′-fluoroanilino)-7-methoxy-6-(3-morpholinopropoxy)quinaquinazoline or a pharmaceutically-acceptable salt thereof (the Agent) from a pharmaceutical composition, which method comprises:
(i) immersing a pharmaceutical composition containing the Agent in a dissolution medium, wherein the dissolution medium comprises water and a non-ionic surfactant; and
(ii) determining the concentration of the Agent in the dissolution medium at one or more time points following immersion of the pharmaceutical composition.

Suitable non-ionic surfactants for use in the dissolution medium include polyoxyethylene sorbitan fatty acid esters, sorbitan fatty acid esters, polyoxyethylene esters, polyoxyethylene ethers, or polyoxyethylene/polyoxypropylene block copolymers (Poloxamers) and mixtures thereof.

In an embodiment of the present invention the non-ionic surfactant is selected from a polyoxyethylene sorbitan fatty acid ester or a mixture thereof. A variety of polyoxyethylene sorbitan fatty acid esters are suitable for use in the present invention. Conveniently the polyoxyethylene sorbitan fatty acid ester is one formed with a polyoxyethylene sorbitan and an aliphatic fatty acid. Suitable aliphatic fatty acids include those with from 10 to 24 carbon atoms, which may saturated or unsaturated fatty acids. Examples of suitable fatty acids include for example, lauric, palmitic, stearic or oleic acid. Often such aliphatic fatty acids are derived form natural sources and comprise a mixture of aliphatic fatty acids. Accordingly, when esters are prepared using a mixture of aliphatic fatty acids a mixture of esters will result. Reference herein to a particular ester, such as a polyoxyethylene sorbitan monolaurate, refers to an ester in which the lauric acid is the predominant fatty acid group present. By “predominant fatty acid” is meant at least 50%, preferably at least 60% and more preferably at least 80% of the acid groups in the surfactant are the same. A similar convention is adopted for the other ester and ether surfactants mentioned herein. The ester may be a mono- or polyester of a polyoxyethylene sorbitan, for example, a mono-, di- or tri-ester, such as polyoxyethylene sorbitan mono-oloeate or polyoxyethylene sorbitan tri-oloeate. Particular polyoxyethylene sorbitan fatty acid esters include a polyoxyethylene sorbitan monolaurate, a polyoxyethylene sorbitan monooleate or a mixture thereof. The number of oxyethylene repeat units in the polyoxyethylene sorbitan fatty acid ester surfactant may be varied over wide limits, for example from 5 to 80. Generally, however from 10 to 30, such as 20 repeat units are preferred. Particular polyoxyethylene sorbitan fatty acid ester surfactants include polyoxyethylene (20) sorbitan monolaurate (commercially available as Tween™ 20 and polyoxyethylene (20) sorbitan monooleate (commercially available as Tween™ 80), wherein “(20)” above refers to the average number of oxyethylene repeat units present. A particularly suitable non-ionic surfactant is polyoxyethylene (20) sorbitan monooleate such as Tween™ 80.

Suitable sorbitan fatty acid esters include mono-, di- and tri-esters of sorbitan with a suitable aliphatic fatty acid or mixture of acids. Suitable aliphatic fatty acids include, for example those described above in relation to the polyoxyethylene sorbitan fatty acid ester surfactants. Particular sorbitan fatty acid esters include sorbitan monolaurate (Span™-20), sorbitan monopalmitate (Span™-40), sorbitan monostearate (Span™-60), sorbitan monooleate (Span™-80), sorbitan trioleate (Span™-85) or a mixture thereof.

Suitable polyoxyethylene esters include esters formed by a suitable aliphatic fatty acid and a polyethylene glycol. Suitable aliphatic fatty acids include, for example those described above in relation to the polyoxyethylene sorbitan surfactants such as lauric, palmitic, stearic or oleic acid or a mixture thereof. The ester may be a mono or di-ester with the polyethylene glycol, but is preferably a monoester. Suitable polyethylene glycols include those with from 1 to 400 oxyethylene repeat units, for example from 1 to 200 oxyethylene repeat units.

Suitable polyoxyethylene ethers are ethers formed between a polyethylene glycol and a aliphatic alcohol or mixture of alcohols. Suitable aliphatic alcohols are those with from 8 to 24 carbon atoms, for example from 10 to 20 carbon atoms. The aliphatic alcohol may be saturated or unsaturated. Particular aliphatic alcohols include, for example, lauryl, cetyl, stearyl or oleyl alcohol or a mixture thereof. Suitable polyethylene glycols are those described above in relation to the polyoxyethylene ester non-ionic surfactants, particularly polyethylene glycols with from 2 to 20 oxyethylene repeat units. Polyoxyethylene ether non-ionic surfactants are commercially available as, for example Brij™ surfactants ex. ICI Ltd.

Suitable polyoxyethylene/polyoxypropylene block copolymers comprise A-B-A block co-polymers in which A represents a polyoxyethylene block and B a polyoxypropylene block. Such polymers are widely available as “Poloxamer” surfactants such as Synperonic™ (ex ICI Ltd), Pluronic™ (ex BASF).

Suitably the non-ionic surfactant is present in the dissolution medium at a concentration greater than or equal to critical micelle concentration (CMC) of the non-ionic surfactant. For example, the dissolution medium contains at least 0.001% v/v, particularly at least 0.01% v/v, more particularly at least 0. 1% v/v non-ionic surfactant. In an embodiment of the invention the non-ionic surfactant is present in the dissolution at a concentration in excess of the CMC of the surfactant, for example 10, 20 or 30 times the CMC of the surfactant. We have surprisingly found that relatively high surfactant concentrations in the dissolution medium provides a high degree of discrimination between different pharmaceutical formulations containing the Agent. Accordingly in this embodiment a particular non-ionic surfactant concentration in the at least 1% v/v, for example 2.0, 2.5, 3, 3.5, 4, 4.5, 5, 5.5, 6, 6.5, 7, 7.5 or 8% v/v. The upper limit of non-ionic surfactant concentration will vary depending upon the type of non-ionic surfactant used. In general, we have found that increasing beyond an optimum upper level provides no significant improvement in the release of the Agent of the ability of the method according to the invention to discriminated between different formulations or batches. Furthermore, further increases in non-ionic surfactant concentration may result in a poor in-vitro to in-vivo correlation. Generally, an upper limit of non-ionic surfactant of 4, 4.5, 5, 5.5, 6, 6.5, 7, 7.5, 8, 8.5 or 9% v/v is sufficient. Generally the concentration of non-ionic surfactant may be from 0.001 to 8.0% v/v, for example from 0.5 to 8.0% v/v, particularly from 2.0 to 8.0% v/v, more particularly from 2 to 6% v/v and still more particularly from 4 to 6% v/v. In another embodiment the non-ionic surfactant is Tween™ 80 and the concentration of non-ionic surfactant is about 5% v/v (for example from 4.5 to 5.5% v/v). We have found that a concentration of about 5% Tween™ 80 provides particularly good discrimination between different pharmaceutical compositions containing the Agent. In a further particular embodiment the non-ionic surfactant is Tween™ 20 and is present in the dissolution medium at a concentration of about 4% v/v (for example from 3.5 to 4.5% v/v).

In another embodiment, the pH of the dissolution medium is from 6.0 to 8.0. We have found that at a pH of less than 6.0 the discrimination between different formulations was significantly reduced. At pH greater than 6.0 the discrimination between different formulations tested increased, however, as pH increases the solubility of the Agent decreases significantly and above a pH of 8.0 it is likely that the solubility and dissolution rate of the Agent would be too low to carry out the dissolution test in a reasonable time. In an embodiment of the present invention the pH of the dissolution medium is from 6.5 to 8.0, more particularly from 7.0 to 8.0 and still more particular 7.0 to 7.5.

The dissolution medium is optionally buffered to maintain the pH at a required level. Suitable buffers are well known and include, for example a phosphate buffer. However, we have found that for many formulations containing the Agent a buffer is not required because the pH of the dissolution medium does not change significantly (for example less than 0.5 pH units) during the method according to the invention. Furthermore, we have surprisingly shown that when the non-ionic surfactant is a polyoxyethylene sorbitan fatty acid ester such as a Tween™ surfactant, particularly Tween™ 20 or Tween™ 80 and the dissolution medium is un-buffered, that the method gives improved dissolution and better discrimination between batches/different formulations of the Agent compared to the same test using a buffer such as a phosphate buffer.

The dissolution medium is suitably maintained at a constant temperature, for example within a range of ±1° C., preferably ±0.5° C. Suitably the temperature of the dissolution medium is representative of temperatures found in-vivo, for example from 34 to 42° C. In an embodiment the temperature of the dissolution medium is about 37° C, for example 37±0.5° C. The temperature of the dissolution medium may be maintained at a constant level using conventional means, for example by placing a vessel containing the dissolution medium in a thermostatically controlled water bath at the desired temperature.

Suitably the water used to prepare the dissolution medium is deionized water. The deionized water is optionally degassed prior to use in the method of the invention as described in the USP 24. However, we have surprisingly found that the method according to the present invention provides a high degree of repeatability and remains discriminatory between different formulations of the Agent even when using deionized water that has not been degassed.

Optionally the dissolution medium may contain additional components that control or enhance dissolution of the Agent, for example additives to control the ionic strength (or osmolarity) of the dissolution medium and/or additives to adjust pH to the required level. However, it is preferred that the dissolution medium consists essentially of water and the non-ionic surfactant.

The volume of dissolution medium used in the present method should be sufficient to ensure that all of the Agent contained in the pharmaceutical composition being tested can be dissolved in the dissolution medium without saturating the dissolution medium. The Agent is classified under the Biopharmaceutics Classification System (BCS) as a Class II compound (Amidon et al., Pharm. Res. 1995, 12:413-420). Under the BCS system Class II compounds are sparingly soluble with high permeability. In view of the BCS classification of the Agent it is preferred that the dissolution medium is present in sufficient volume to provide sink conditions in accordance with the guidelines in USP 24. By “sink conditions” is meant that more than three times the volume of dissolution medium required to form a saturated solution of the Agent is present. However, we have found that true sink conditions are not essential and that a volume sufficient to provide a concentration of Agent in the dissolution medium of less than 1 mg/ml following complete dissolution of the Agent in the dissolution medium is sufficient, for example a final concentration of Agent of from 0.5 to 1 mg/ml, such as from 0.5 to 0.75 mg/ml. Generally for pharmaceutical compositions containing 250 mg of the Agent, approximately 500 to 4000 ml of dissolution medium is suitable, for example 500, 900, 1000, 2000 or 4000 ml.

The pharmaceutical composition containing the Agent should be immersed in the dissolution medium such that it is fully covered by the dissolution medium. This minimises the variability between runs of the method according to the invention.

Suitable apparatus which may be used for carrying out the method of the present invention include, but are not limited to, known dissolution apparatus, for example those described in USP 24 such as Apparatus 1 (basket test), Apparatus 2 (paddle test) and modifications thereof. These apparatus are well known to those of ordinary skill in the art or by reference to the USP which is incorporated herein by reference. A particularly suitable apparatus is the USP 24 Apparatus 2 which comprises a dissolution vessel containing a paddle assembly. The paddle is rotated slowly to provide gentle agitation of the dissolution medium. Suitable rotation speeds are typically up to 100 revolutions per minute (rpm), for example from 10 to 100 rpm, such 40 to 100, particularly at about 50 rpm, for example 50±1 rpm.

As will be clear the term “release of Agent” in step (i) of the method according to the invention refers to dissolution of the Agent from the pharmaceutical composition into the dissolution medium. Accordingly in step (ii) of the process it is convenient to determine the concentration of Agent is solution in the dissolution medium as a % of the total Agent present in the pharmaceutical composition.

In step (ii) of the method according to the invention the concentration of the Agent in the dissolution medium is determined at one or more time points following immersion of the pharmaceutical composition in the dissolution medium. Determining the concentration at a plurality of time points enables the dissolution profile to be measured over time. However, for quality control purposes it is often sufficient to measure the concentration of agent at a single set time point and compare the measured concentration to a previously determined reference value or concentration range. A deviation from the required specification thereby enables unacceptable products or batches to be quickly and efficiently identified. Generally, for initial studies on a new formulation or to characterise the effects of, for example storage, on a formulation it is desirable to obtain a dissolution profile over time to monitor for changes at any point during the dissolution. The number of concentration measurements over time will depend upon the characterisation that is required. Generally a concentration measurement every 5 to 20 minutes provides sufficient information to accurately assess the dissolution profile. However longer or shorter measurement times may be appropriate, for example in embodiments the concentration of Agent in the dissolution medium may be monitored constantly.

The concentration of Agent in the dissolution medium may be measured using conventional analytical methods well known to those of ordinary skill in the art, for example high performance liquid chromatography (HPLC) or ultraviolet analysis using a suitable spectrophotometer. The particular analytical method selected will be determined largely by the concentration of the Agent and the nature and quantities of the excipients present in the formulation being tested. In one embodiment of the invention a small sample of the dissolution medium (for example 5-20 ml) is removed at each required time point and the concentration of Agent in each sample is measured to thereby provide a dissolution profile for the pharmaceutical composition containing the agent. The absorbance of sample solutions n₁, n₂, n₃, . . . nᵣ, . . . nₓ is A₁, A₂, A₃ . . . Aᵣ . . . Aₓ where nᵣ is before nₓ and Aᵣ is absorbance at time point nᵣ which is earlier than nₓ are measured using a UV spectrometer. The % dissolution is then given by:

$$\% dissolution = \frac{⌊ {A}_{x} \times F \times ( {V}_{T} - {V}_{S} ) + {V}_{S} \times F \times {\sum }_{r = 1}^{r = x - 1} {A}_{r} ⌋}{N} \times 100$$

where:
% dissolution is the cumulative percentage of nominal drug content release by time point x
Aₓ=absorbance at time point nₓ
Aᵣ=absorbance at time point nr which is earlier than nₓ
F=absorbance of 1 mg of the Agent in 1 ml of dissolution medium
V_{T}=total volume of the dissolution medium, in ml
V_{S}=sample volume, in ml
N=nominal content, in mg, of the Agent in the formulation being tested.
In another embodiment samples may be taken from the dissolution medium and fresh dissolution medium is added such that the total volume of dissolution medium remains constant throughout the test. In this embodiment the above equation is modified appropriately.
   The method according to the invention may be used to measure release of the Agent from a wide variety of pharmaceutical compositions. The method is particularly suitable for use with solid pharmaceutical compositions containing the Agent, for example solid immediate release pharmaceutical compositions suitable for oral administration containing the Agent, such as tablet or capsule formulations containing the Agent, although other pharmaceutical compositions may be used. By “immediate release” is meant a composition that releases the Agent as soon as the pharmaceutical composition is administered to a patient and wherein substantially all of the Agent is released within a short period of time, typically less than 60 minutes.

In an embodiment of the method according to the present invention there is provided

a method for measuring the release of Agent from a solid pharmaceutical composition, which method comprises:

(i) immersing a pharmaceutical composition containing the Agent in a dissolution medium, wherein the dissolution medium comprises water and from 2 to 8% v/v (particularly from 4 to 6% v/v) of a non-ionic surfactant selected from a polyoxyethylene sorbitan monolaurate (such as Tween™ 20) and a polyoxyethylene sorbitan monooleate (such as Tween™ 80),

wherein the pH of the dissolution medium is from 6.0 to 8.0 (particularly from 7.0 to 8.0); and
(ii) determining the concentration of the Agent in the dissolution medium at one or more time points following immersion of the pharmaceutical composition.

In this embodiment a particular non-ionic surfactant is a polyoxyethylene sorbitan monooleate (more particularly Tween™ 80).

The method of the present invention is particularly useful for monitoring batch-to-batch variability as part of a quality control procedure; as a means for determining the bioequivalence of different formulations; and as a means for assessing the durability of a formulation (for example the storage stability).

To determine, for example, bioequivalence between different compositions containing the Agent, the dissolution profile obtained using the method according the present invention is compared to a reference dissolution profile to assess whether or not the measured formulation meets the required specification based upon the reference dissolution profile. The reference dissolution profile will usually be one that has been generated using a pharmaceutical composition that has also been characterised in-vivo, thereby enabling a correlation to be made between the in-vitro data generated by the method of the invention with the required in-vivo profile. Conveniently the reference dissolution profile used to assess the new composition will be a dissolution profile generated using the method according to the present invention so that a direct comparison of the two in-vitro profiles from each composition can be made. If the dissolution profile obtained from the new composition is within acceptable limits of the reference dissolution profile the two compositions are considered bioequivalent.

Accordingly a further aspect of the present invention provides a method for determining the bioequivalence of a first pharmaceutical composition containing the Agent to a reference pharmaceutical composition containing the Agent comprising:
(a) measuring the release of Agent from the first and reference pharmaceutical compositions using the method according to the first aspect of the present invention; and
(b) comparing the release of Agent from the first pharmaceutical composition with the release of Agent from the reference pharmaceutical composition.
   The invention is illustrated below by the following non-limiting examples, wherein the Agent is the free base form of the compound of formula I.

### IN THE FIGURES:

FIG. 1 is a plot of % release of the Agent from two different tablet formulations, Tablet A and Tablet B in a dissolution medium containing 5% v/v Tween™-80. In FIG. 1 the triangular data points represent Tablet A and the square data points represent Tablet B. The error bards in FIG. 1 represent ±2 standard deviations.

FIG. 2 is a plot of % release of the Agent from two different tablet formulations, Tablet A and Tablet B in a dissolution medium containing various quantities of the anionic surfactant sodium lauryl sulfate. In FIG. 2 the triangular data points represent release of Agent from the two tablets in a dissolution medium containing 1% sodium lauryl sulfate. The diamond shaped data points represent release of Agent from the two tablets in a dissolution medium containing 0.25% v/v sodium lauryl sulfate.

### PHARMACEUTICAL COMPOSITIONS CONTAINING THE AGENTS

Two similar film coated tablet formulations, Tablet A and Tablet B containing 250 mg of the Agent were prepared using slightly different wet granulation, direct compression and film coating techniques:

Tablet A

| | |
|---|---|
| Tablet core | |
| The Agent | 250.0 mg |
| Lactose monohydrate | 163.5 mg |
| Microcrystalline cellulose | 50.0 mg |
| Croscarmellose sodium | 20.0 mg |
| Povidone | 10.0 mg |
| Sodium lauryl sulfate | 1.5 mg |
| Magnesium stearate | 5.0 mg |
| Tablet coating | |
| Hydroxypropyl methylcellulose¹ | 7.65 mg |
| Polyethylene glycol 300 | 1.5 mg |
| Titanium Dioxide | 0.50 mg |
| Yellow ferric oxide | 0.90 mg |
| Red ferric oxide | 0.90 mg |

Tablet B

| | |
|---|---|
| Tablet core | |
| The Agent | 250.0 mg |
| Lactose monohydrate | 163.5 mg |
| Microcrystalline cellulose | 50.0 mg |
| Croscarmellose sodium | 20.0 mg |
| Povidone | 10.0 mg |
| Sodium lauryl sulfate | 1.5 mg |
| Magnesium stearate | 5.0 mg |
| Tablet coating | |
| Hydroxypropyl methylcellulose¹ | 8.16 mg |
| Polyethylene glycol 300 | 1.60 mg |
| Talc | 1.18 mg |
| Titanium Dioxide | 1.18 mg |
| Yellow ferric oxide | 0.04 mg |

Footnote
¹Grade 2910, 6 cp viscosity (measured at 2% w/v at 20° C.) ex Shin Etsu).

In-Vivo Profiles of Tablets A and B

An open, randomised, 2-way cross-over comparison of Tablet A and Tablet B in healthy volunteers was carried out to determine the pharmacokinetic properties of the two tablets, primarily the Area under the curve (AUC) and peak plasma concentration of the Agent.

Method

Design: A randomised, open-label, 2-way cross-over, Phase I trial. During the first period (Period 1), volunteers received a single oral dose of either 1×250 mg Agent formulated as Tablet A, or 1×250 mg Agent formulated as Tablet Bormulation tablet. This was followed by a washout period of at least 3 weeks. During Period 2, volunteers were given the treatment that they did not receive in Period 1.
Key inclusion criteria: Male, aged .18 years; normal clinical examination, including medical history and resting electrocardiogram (ECG); veins suitable for multiple venepunctures.
Key exclusion criteria: Use of regular medication or therapy; acute illness within 2 weeks before the start of the trial; any clinically significant abnormalities in clinical chemistry, haematology, or urinalysis results; definite or suspected personal history or family history of significant adverse drug reactions, or hypersensitivity to drugs with a similar chemical structure to the Agent; history or presence of gastrointestinal, hepatic, or renal disease, or other condition known to interfere with absorption, distribution, metabolism, or excretion of drugs; treatment in the previous 3 months with any drug known to have a well-defined potential for hepatotoxicity.
Key pharmacokinetic assessments: The primary endpoints of this trial were the following pharmacokinetic parameters: area under the plasma concentration-time curve from time 0 to infinity (AUC) and maximum plasma concentration (Cmax ) of the Agent, for the assessment of bioequivalence of Tablet A and Tablet B. The secondary pharmacokinetic endpoints were the area under the plasma concentration-time curve from time 0 to the time of the last quantifiable concentration [AUC(0-t)]; time of maximum plasma concentration (tmax ), slowest disposition rate constant (lz ); and terminal half-life (t½) of the Agent.

The results of this in-vivo study are shown in Table 1

TABLE 1
Statistical analysis of AUC and Cₘₐₓ

| | | Tablet | Tablet | |
|---|---|---|---|---|
| | | A | B | Analysis |
| AUC | Geometric least squares | 2435.0 | 2346.5 | |
| (ng · h/ml) | mean* | | | |
| | N | 35 | 33 | |
| | Estimate of treatment ratio | | | 0.964 |
| | (AUC Tablet B/AUC Tablet | | | |
| | A) | | | |
| | Lower 90% confidence | | | 0.865 |
| | interval | | | |
| | Upper 90% confidence | | | 1.073 |
| | interval | | | |
| Cₘₐₓ | Geometric least squares | 92.4 | 76.7 | |
| (ng/ml) | mean* | | | |
| | N | 36 | 34 | |
| | Estimate of treatment ratio | | | 0.830 |
| | (Cₘₐₓ Tablet B/Cₘₐₓ Tablet | | | |
| | A) | | | |
| | Lower 90% confidence | | | 0.710 |
| | interval | | | |
| | Upper 90% confidence | | | 0.971 |
| | interval | | | |

*geometric least squares mean obtained from the statistical model used to analyse the data

The results in Table 1 show that the AUC for each Tablet formulation A and B was similar. However, the peak plasma concentration, Cₘₐₓ, for Tablet B was lower (about 17%) than that for Tablet A. This in-vivo data clearly shows that Tablets A and B exhibit different in-vivo profiles. An in-vitro dissolution method is therefore required that is able to discriminate between different formulations containing the Agent.

### EXAMPLE 1

A 5% v/v solution of Tween™ 80 (ex Acros Organics) in water was prepared as a dissolution medium by mixing 1 part Tween™ 80 is mixed with 19 parts deionised water using appropriate mixing and quantitative transfer (due to viscosity). If required the pH of the solution was adjusted to 7.0 to 7.5 using a concentrated base or acid such as concentrated HCl as appropriate.

1000 ml of this dissolution medium was placed in a USP 24 <71 1> Apparatus 2 (paddle apparatus). The dissolution medium was stirred using the paddle at rotation rate of 50 revolutions per minute and the temperature of the dissolution medium was controlled to 37±0.5° C. using a heater and circulation pump within, or optionally external, to the vessel containing the dissolution medium.

Tablet A was placed in the dissolution medium such that the entire tablet was covered by the dissolution medium. 10 ml samples of the dissolution medium were then taken at 15, 30, 45 and 60 minutes following immersion of Tablet A.

Each sample of dissolution medium was filtered immediately through a 0.45 μm PTFE syringe filter, discarding at least the first 2 ml of filtrate. The concentration of Agent in each filtered sample was then measured using a UV spectrophotometer (Hewlett Packard 8452D diode array in a 1 mm cell at a wavelength of 334 nm). The % Agent in each sample was determined by comparing the UV analysis to that obtained from a standard solution of the Agent at a concentration representing 100% release of the Agent.

The procedure described above was repeated using Tablet B.

The dissolution test was repeated using a total of 80 of each Tablet A and Tablet B.

Results

The results from the dissolution tests according to the present invention, together with the standard deviation for the concentration of Agent at each time point, is shown in Tables 2 and 3.

TABLE 2
Summary dissolution data (n = 80) for Tablet A in 5%
v/v Tween ™ 80 dissolution medium

| | % Dissolution | | |
|---|---|---|---|
| Time (mins) | Mean | Range | Standard deviation |
| 15 | 54 | 32-63 | 6.7 |
| 30 | 82 | 72-86 | 2.3 |
| 45 | 90 | 87-93 | 1.4 |
| 60 | 94 | 91-97 | 1.4 |

TABLE 3
Summary dissolution data (n = 80) for Tablet B in 5%
v/v Tween ™ 80 dissolution medium

| | % Dissolution | | |
|---|---|---|---|
| Time (mins) | Mean | Range | Standard deviation |
| 15 | 51 | 38-59 | 4.5 |
| 30 | 71 | 65-74 | 2.1 |
| 45 | 79 | 74-82 | 1.9 |
| 60 | 84 | 80-87 | 1.7 |

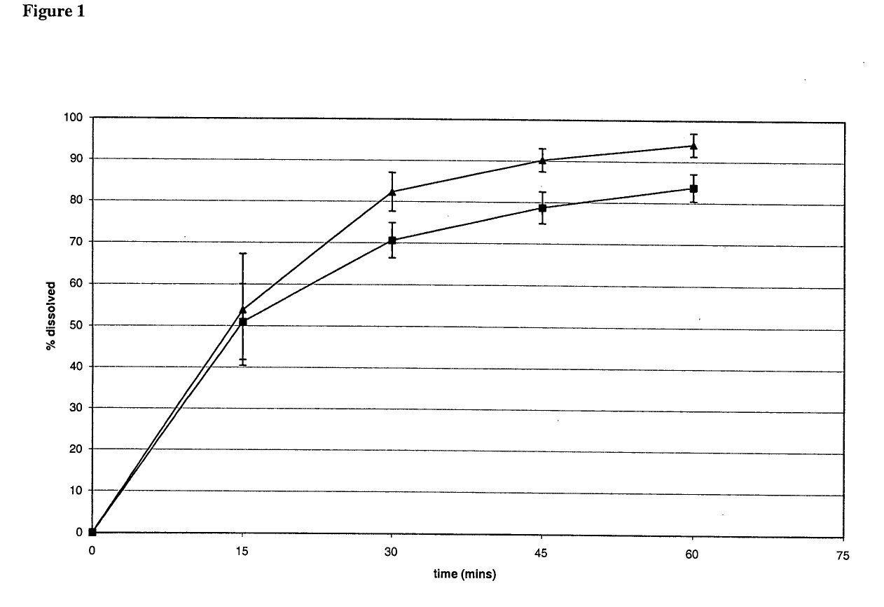

The data in Tables 2 and 3 is plotted in FIG. 1 in which the triangular points represent data from Tablet A, the square points data from Tablet B and the error bars indicate ±2× the standard deviation for each point shown in Tables 2 and 3 .

The data clearly shows that the method according to the present invention is able to differentiate between the two tablet formulations and therefore predict that Tablet A and Tablet B will not exhibit the same in-vivo profiles. In particular clear differentiation is obtained between the two dissolution profiles at the 30, 45 and 60 minute sampling points.

### EXAMPLE 2

### Effect Surfactant Concentration

The method described in Example 1 was repeated using different concentrations of Tween™ 80 in the dissolution medium for both Tablet A and Tablet B. The concentration of Agent for each sample point is shown in Tables 4 and 5. In Tables 4 and 5 the concentration of surfactant used for each test is shown in the first row. The value of “n” refers to the number of tests carried out on each of the two tablet formulations, A and B.

The difference between the mean concentrations of Agent measured for Tablet A and Tablet B at each time point is shown in Table 6.

TABLE 4
Dissolution Results (Mean % Agent Release) for Varying Tween ™
80 Concentrations from Tablet A

| Time | Tween ™ 80 Concentration | | | | | |
|---|---|---|---|---|---|---|
| (minutes) | 2% (n = 2) | 3% (n = 2) | 4% (n = 2) | 5% (n = 2) | 5% (n = 6) | 6% (n = 6) |
| 15 | 48 | 46 | 52 | 58 | 49 | 56 |
| 30 | 66 | 71 | 77 | 82 | 79 | 84 |
| 45 | 73 | 79 | 85 | 90 | 88 | 91 |
| 60 | Not | 84 | 90 | 93 | 92 | 94 |
| | measured | | | | | |

TABLE 5
Dissolution Results (Mean % Agent Release) for Varying Tween ™
80 Concentrations Tablet B

| Time | Tween ™ 80 Concentration | | | | | |
|---|---|---|---|---|---|---|
| (minutes) | 2% (n = 2) | 3% (n = 2) | 4% (n = 2) | 5% (n = 2) | 5% (n = 6) | 6% (n = 6) |
| 15 | 45 | 47 | 51 | 52 | 47 | 51 |
| 30 | 58 | 62 | 66 | 68 | 69 | 71 |
| 45 | 64 | 69 | 74 | 78 | 76 | 80 |
| 60 | Not | 74 | 79 | 83 | 81 | 86 |
| | measured | | | | | |

TABLE 6
Difference in mean dissolution results (Tablet A − Tablet B) for
varying Tween ™ 80 Concentrations

| Time | Tween ™ 80 Concentration | | | | | |
|---|---|---|---|---|---|---|
| (minutes) | 2% (n = 2) | 3% (n = 2) | 4% (n = 2) | 5% (n = 2) | 5% (n = 6) | 6% (n = 6) |
| 15 | 3 | −1 | 1 | 6 | 2 | 5 |
| 30 | 8 | 9 | 11 | 14 | 11 | 12 |
| 45 | 9 | 10 | 11 | 12 | 13 | 10 |
| 60 | Not | 9 | 11 | 10 | 11 | 8 |
| | measured | | | | | |

Results

The data in Tables 4 to 6 show that the method according to the present invention was able to discriminate between Tablets A and B for surfactant concentrations of from 2 to 6% v/v. Table 4 shows that the optimum discrimination occurs with a dissolution medium containing 5% v/v Tween™ 80. The optimum time point for discriminating between Tablet A and Tablet B occurs between about 30 and 45 minutes after immersion of the tablet in the dissolution medium.

### EXAMPLE b 3

### Dissolution in a Dissolution Medium Containing Tween™ 20

The method described in Example 1 was repeated using different concentrations of Tween™ 20 in the dissolution medium for both Tablet A and Tablet B. The concentration of Agent for each sample point is shown in Tables 7 and 8. In Tables 7 and 8 the concentration of Tween™ 20 surfactant used for each test is shown in the first row. The value of “n” refers to the number of tests carried out on each of the two tablet formulations A and B.

The difference between the mean concentrations of Agent measured for Tablet A and Tablet B at each time point is shown in Table 9.

TABLE 7
Dissolution Results (Mean % Agent Release) for Varying
Tween ™ 20 Concentrations Tablet A

| | Tween ™ 20 Concentration | | | |
|---|---|---|---|---|
| Time (minutes) | 2% (n = 3) | 3% (n = 3) | 4% (n = 2) | 5% (n = 2) |
| 15 | 48 | 56 | 51 | 54 |
| 30 | 69 | 77 | 79 | 81 |
| 45 | 75 | 84 | 87 | 88 |
| 60 | 79 | 88 | 91 | 92 |

TABLE 8
Dissolution Results (Mean % Agent Release) for Varying
Tween ™ 20 Concentrations Tablet B

| | Tween ™ 20 Concentration | | | |
|---|---|---|---|---|
| Time (minutes) | 2% (n = 3) | 3% (n = 3) | 4% (n = 2) | 5% (n = 2) |
| 15 | 50 | 56 | 49 | 56 |
| 30 | 61 | 67 | 67 | 71 |
| 45 | 67 | 74 | 75 | 78 |
| 60 | 71 | 79 | 80 | 83 |

TABLE 9
Difference in mean dissolution results (Tablet A − Tablet B) for
varying Tween ™ 20 Concentration

Tween ™
20 Concentration

| Time (minutes) | 2% | 3% | 4% | 5% |
|---|---|---|---|---|
| 15 | 2 | 0 | 2 | −2 |
| 30 | 8 | 10 | 12 | 10 |
| 45 | 8 | 10 | 12 | 10 |
| 60 | 8 | 9 | 11 | 9 |

Results

The results in Tables 7 to 9 clearly indicate that a dissolution medium containing Tween™ 20 is able to discriminate between Tablets A and B. Table 9 shows that the optimum concentration of Tween™ 20 was about 4% v/v. Table 9 also indicates that the optimum discrimination between Tablets A and B occurs between about 30 and 45 minutes. For example at 45 minutes following immersion the method according to the invention detected a 12% difference in the % agent released from Tablet A compared to Tablet B.

### COMPARATIVE EXAMPLE 1

### Dissolution in Acidic Dissolution Media

The method described in Example 1 was repeated but using a standard USP acidic dissolution medium consisting of a buffered aqueous dissolution medium with a pH of 1.2, using a hydrochloric acid/potassium chloride buffer (similar to the 0.1 N buffered HCl described in 24 USP). The concentration of Agent measured at 15, 30 and 45 minutes for Tablets A and B are shown in Table 10.

TABLE 10
Dissolution data (% Agent release) in pH 1.2 buffer dissolution
medium

| | Tablet A (n = 6) | | Tablet B (n = 6) | | Difference in |
|---|---|---|---|---|---|
| Time (mins) | Mean | Range | Mean | Range | dissolution |
| 15 | 99 | 90-103 | 101 | 100-102 | −2 |
| 30 | 100 | 91-103 | 101 | 101-102 | −1 |
| 45 | 100 | 91-103 | 102 | 101-103 | −2 |

Results

Table 10 shows that more than 85% dissolution was achieved in 15 minutes at pH 1.2. However, despite the high solubility of the Agent in this acidic dissolution medium, there was no measurable difference between the dissolution profiles of Tablets A and B as shown by the final column in Table 10.

These results clearly show that this standard acidic dissolution medium would incorrectly predict that Tablet A and Tablet B would have the same in-vivo profile.

### COMPARATIVE EXAMPLE 2

### Dissolution in Simulated Intestinal Fluid

Example 1 was repeated using 500 ml of simulated fasted intestinal fluid (FaSSIF, pH 6.5 as described in Dressman, et al Eur. J. Pharm. Sci. 11 Suppl 2: S73-S80, 2000) as the dissolution medium. The primary site of absorption of the Agent is thought to be in the intestine. It was, therefore, expected that the use of a dissolution medium that simulated the in-vivo dissolution medium at the site of absorption of the Agent would be sensitive to small batch-to-batch variations/formulation modifications.

However, no measurable difference was observed between the dissolution profiles for Tablet A and Tablet B. Furthermore, only 4% of the Agent had been dissolved in 1 hour.

These results clearly indicate that simulated intestinal fluid as a dissolution medium cannot distinguish between different pharmaceutical compositions containing the Agent. The use of such a medium would falsely suggest that Tablet A and Tablet B would exhibit the same in-vivo profile.

### COMPARATIVE EXAMPLE 3

### Dissolution in aqueous Sodium Lauryl Sulfate

The method described in Example 1 was repeated but using aqueous solutions of sodium lauryl sulfate (SLS) with concentrations of from 0.25 to 1.0% SLS as the dissolution medium. The initial pH of the medium was about 7.0, however, during the test the pH of the medium generally increased to about 8. The concentration of Agent measured at each sample point for the various dissolution media tested are shown in Table 11.

TABLE 11
Dissolution data (% Agent release) from Tablet A and Tablet B in
different concentrations of sodium lauryl sulfate

| | 1% SLS (n = 2) | | 0.5% SLS (n = 2) | | | |
|---|---|---|---|---|---|---|
| Time | Tablet | Tablet | Tablet | | 0.25% SLS (n = 2) | |
| (mins) | A | B | A | Tablet B | Tablet A | Tablet B |
| 15 | 93 | 91 | 86 | 83 | 50 | 49 |
| 30 | 101 | 99 | 98 | 95 | 60 | 57 |
| 45 | 101 | 101 | 100 | 99 | 62 | 60 |
| 60 | 102 | 102 | 101 | 101 | 63 | 62 |

Results

The data in Table 11 shows that approximately all of the Agent was dissolved in 30 minutes for concentrations of SLS>0.5%. However, despite the high solubility in this dissolution medium, a comparison between the concentration of Agent released from each Tablet (A and B) at a given time point shows that there is no significant difference in release of Agent.

Table 11 clearly illustrates that the use of a dissolution medium containing SLS fails to discriminate between different pharmaceutical formulations containing the Agent.

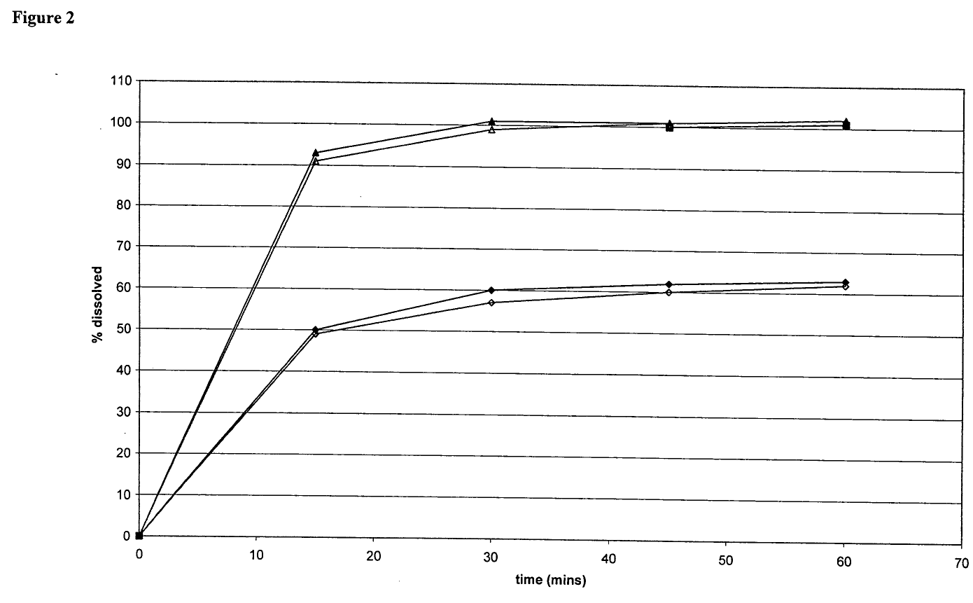

The use of such a dissolution medium would falsely predict that Tablets A and B would be expected to have the same in-vivo dissolution profile and DMPK behaviour. This is clearly shown in FIG. 2 which shows that the use of various concentrations of SLS failed to detect a significant difference in dissolution profiles between Tablet A and Tablet B.

1. A method for measuring the release of 4-(3′-chloro-4′-fluoroanilino)-7-methoxy-6-(3-morpholinopropoxy)quinazoline or a pharmaceutically acceptable salt thereof, (the Agent) from a pharmaceutical composition, which method comprises:
(i) immersing a pharmaceutical composition containing the Agent in a dissolution medium, wherein the dissolution medium comprises water and a non-ionic surfactant; and
(ii) determining the concentration of the Agent in the dissolution medium at one or more time points following immersion of the pharmaceutical composition.

2. The method according to claim 1, wherein the non-ionic surfactant is a polyoxyethylene sorbitan fatty acid ester or a mixture thereof.

3. The method according to claim 1 or claim 2, wherein the non-ionic surfactant is selected from a polyoxyethylene sorbitan monolaurate and a polyoxyethylene sorbitan monooleate.

4. The method according to claim 3 wherein the non-ionic surfactant is a polyoxyethylene sorbitan monooleate.

5. The method according to claim 1 wherein the dissolution medium contains at least 2% v/v non-ionic surfactant.

6. The method according to claim 5, wherein the dissolution medium contains from 2 to 8% v/v non-ionic surfactant.

7. The method according to claim 5, wherein the dissolution medium contains from 4 to 6% v/v non-ionic surfactant.

8. The method according to claim 1, wherein the pH of the dissolution medium is from 6.0 to 8.0.

9. The method according to claim 8 wherein the pH of the dissolution medium is from 7.0 to 8.0.

10. The method according to claim 1, wherein the temperature of the dissolution medium is about 37° C.

11. The method according to claim 1, for measuring the release of Agent from a solid pharmaceutical composition, which method comprises:
(i) immersing a pharmaceutical composition containing the Agent in a dissolution medium, wherein the dissolution medium comprises water and from 2 to 8% v/v of a non-ionic surfactant selected from a polyoxyethylene sorbitan monolaurate and a polyoxyethylene sorbitan monooleate,
wherein the pH of the dissolution medium is from 6.0 to 8.0; and
(ii) determining the concentration of the Agent in the dissolution medium at one or more time points following immersion of the pharmaceutical composition.

12. The method according to claim 11, wherein the non-ionic surfactant is Tween™ 80 and the pH of the dissolution medium is from 7.0 to 8.0.

13. The method according to claim 12, wherein the concentration of Tween™ 80 in the dissolution medium is about 5% v/v

14. The method according to claim 1, wherein the volume of the dissolution medium is selected to give a concentration of Agent in the dissolution medium of less than 1 mg/ml upon complete dissolution of the Agent in the dissolution medium from the pharmaceutical composition containing the Agent.

15. The method according to claim 1, wherein the pharmaceutical composition is a solid immediate release pharmaceutical composition.

16. (canceled)

17. A method for determining the bioequivalence of a first pharmaceutical composition containing the Agent to a reference pharmaceutical composition containing the Agent comprising:
(a) measuring the release of Agent from the first and reference pharmaceutical compositions using the method according to claim 1; and
(b) comparing the release of Agent from the first pharmaceutical composition with the release of Agent from the reference pharmaceutical composition.

18. A method for determining the bioequivalence of a first pharmaceutical composition containing the Agent to a reference pharmaceutical composition containing the Agent comprising:
(a) measuring the release of Agent from the first and reference pharmaceutical compositions using the method according claim 11; and
(b) comparing the release of Agent from the first pharmaceutical composition with the release of Agent from the reference pharmaceutical composition.
